(19) United States
(12) Patent Application Publication
(10) Pub. No.: US 2025/0108099 A1
(43) Pub. Date: Apr. 3, 2025

(54) Ovomucoid for use in a method of managing blood coagulation

(71) Applicant: Solution Shop AG, Basel (CH)

(72) Inventor: Birgit Felser, Basel (CH)

(21) Appl. No.: 18/725,818

(22) PCT Filed: Jan. 3, 2023
(86) PCT No.: PCT/EP2023/050040

§ 371 Date: Jul. 1, 2024

(30) Foreign Application Priority Data
Jan. 4, 2022 (EP) 22150173.7

(51) Int. Cl. A61K 38/57; A61P 7/04

(52) U.S. Cl. CPC A61K 38/57; A61P 7/04

(57) ABSTRACT

The invention provides a novel plasmin inhibitor for use in a method of managing and/or regulating blood coagulation, in particular in managing or treating hyperfibrinolysis.

### BACKGROUND OF THE INVENTION

The fibrinolysis system is responsible for removing blood clots. Hyperfibrinolysis describes a situation with markedly enhanced fibrinolytic activity, resulting in increased and sometimes life-threatening bleeding.

As such, efficient ways to treat and/or prevent hyperfibrinolyis are needed.

Hyperfibrinolysis can be caused by acquired or congenital reasons. Among the congenital conditions for hyperfibrinolysis, deficiency of alpha-2-antiplasmin (alpha-2-plasmin inhibitor) or plasminogen activator inhibitor type 1 (PAI-1) are very rare. The affected individuals show a hemophilia-like bleeding phenotype. Acquired hyperfibrinolysis is found in liver disease, in patients with severe trauma, during major surgical procedures, and other conditions. A special situation with temporarily enhanced fibrinolysis is thrombolytic therapy with drugs which activate plasminogen, e.g. for use in acute ischemic events or in patients with stroke. In patients with severe trauma, hyperfibrinolysis is associated with poor outcome. Moreover, hyperfibrinolysis may be associated with blood brain barrier impairment, a plasmin-dependent effect due to an increased generation of bradykinin.

Treatment or prophylaxis of hyperfibrinolyis is made with synthetic drugs, such as tranexamic acid, epsilon-aminocaproic acid or other lysine analogues with the goal to avoid blood transfusions. These drugs are in general either unspecific or show serious side effects, which is why several treatment options have been abandoned.

There is therefore a need for further and new treatment and/or prevention options for hyperfibrinolysis.

### SUMMARY OF THE INVENTION

The present invention relates to an ovomucoid for use in a method of managing and/or regulating blood coagulation, in particular in a method of preventing and/or treating hyperfibrinolysis associated bleeding.

The invention further relates to a pharmaceutical composition comprising an ovomucoid and a method for managing and/or regulating blood coagulation, comprising administering an ovomucoid to a patient in need thereif.

### DESCRIPTION OF THE FIGURES

FIG. 1 depicts the Thrombin generation in pooled plasma samples comprising 0.5 pM tissue factor (TF) in the absence of fibrinogen under the influence of duck egg ovomucoid (50-350 U/ml) (FIG. 1A), Trasylol (50-350 U/ml) (FIG. 1B) and Cyklokapron (1-8 mg/ml) (FIG. 1C).

FIG. 2 depicts the Thrombin generation in pooled plasma samples comprising 0.5 pM tissue factor (TF) in the absence of fibrinogen under the influence of duck egg ovomucoid (50-350 U/ml) (FIG. 2A), Trasylol (50-350 U/ml) (FIG. 2B) and Cyklokapron (1-8 mg/ml) (FIG. 2C).

FIG. 3 depicts the Thrombin generation in pooled plasma samples comprising 5 pM tissue factor (TF) in the presence of endogenous fibrinogen under the influence of duck egg ovomucoid (50-350 U/ml) (FIG. 3A), Trasylol (50-350 U/ml) (FIG. 3B) and Cyklokapron (1-8 mg/ml) (FIG. 3C).

FIG. 4 depicts the Thrombin generation in pooled plasma samples comprising 0.5 pM tissue factor (TF) in the presence of endogenous fibrinogen under the influence of duck egg ovomucoid (50-350 U/ml) (FIG. 4A), Trasylol (50-350 U/ml) (FIG. 4B) and Cyklokapron (1-8 mg/ml) (FIG. 4C).

FIG. 5 depicts plasmin activity in the presence of varying concentrations of duck egg ovomucoid (FIG. 5A) and Cycklokapron (FIG. 5B)

### DETAILED DESCRIPTION OF THE INVENTION

The present invention relates to an ovomucoid for managing and/or regulating blood coagulation, a new pharmaceutical composition for managing and/or regulating blood coagulation and a method for managing and/or regulating blood coagulation. The composition and method are particularly useful for treatment and/or prevention of hyperfibrinolysis.

The pharmaceutical composition according to the invention comprises a trypsin inhibitor, in particular an ovomucoid for managing and/or regulating blood coagulation. The method comprises administering said pharmaceutical composition to a subject in need thereof.

Accordingly, in a first aspect, the invention relates to a pharmaceutical composition comprising a trypsin inhibitor for use in a method of managing or regulating blood coagulation in a subject. In preferred embodiments of the invention managing or regulating blood coagulation is achieved by modifying, regulating or inhibiting plasmin activity. As such, in some embodiments the trypsin inhibitor is able to inhibit or modify plasmin activity.

It was surprisingly found that ovomucoid, in particular duck egg ovomucoid, is a suitable plasmin inhibitor and may be used in a composition according to the invention.

Ovomucoids are proteins found in egg whites and belong to the class of trypsin inhibitors. The best characterized ovomucoids are chicken egg ovomucoid and turkey egg ovomucoid. Duck egg ovomucoid is known, but not as well characterized as chicken egg or turkey egg ovomucoid.

Duck egg ovomucoid has been analyzed in the past, however, it was found that duck egg ovomucoid does not inhibit human plasmin; see Vlaueva, T. A, et al., Immunopharmacology Volume 32, Issues 1-3, May 1996, Pages 108-110. The inventor surprisingly found that duck egg ovomucoid is a suitable plasmin inhibitor and may be used in a composition to manage and/or regulate blood coagulation, in particular to prevent or treat hyperfibrinolysis.

As such, the composition is particularly suitable for modifying fibrinolysis and in particular suitable for preventing and/or treating hyperfibrinolysis. Therefore, in some embodiments of the invention, managing or regulating blood coagulation refers to treatment and/or prevention of hyperfibrinolysis associated bleeding.

Accordingly, in one embodiment of the invention the trypsin inhibitor is an ovomucoid or a fragment thereof. In particular embodiments the ovomucoid or fragment thereof is selected from chicken egg ovomucoid, turkey egg ovomucoid and duck egg ovomucoid.

In some embodiments the ovomucoid is a full length protein, preferably the full length ovomucoid is selected from chicken egg ovomucoid, turkey egg ovomucoid and duck egg ovomucoid.

In particular preferred embodiments, the ovomucoid or fragment therof is duck egg ovomucoid. The ovomucoid in question may be extracted from egg, or may be recombinantly produced.

It was surprisingly found that duck egg ovomucoid is particularly suitable as plasmin inhibitor to be used in magaging and/or regulation blood coagulation in a subject, without affecting the blood clotting system. Other known drugs used for managing and/or regulating blood coagulation in a subject such as Trasylol or Cyklokapron affect other aspects of the clotting system. In particular, a composition comprising duck egg ovomucoid is particularly useful in the to treatment and/or prevention of hyperfibrinolysis associated bleeding.

Accordingly, in a particular aspect, the invention relates to a composition comprising an ovomucoid for use in a method of managing or regulating blood coagulation in a subject, wherein managing or regulating blood coagulation refers to treatment and/or prevention of hyperfibrinolysis associated bleeding.

In a particular embodiment of the invention, the composition comprises duck egg ovomucid for use in the treatment and/or prevention of hyperfibrinolysis associated bleeding.

The pharmaceutical composition may be administered in any suitable form. Preferably, the composition according to the present invention is a composition for intravenous administration. However, administration of the composition may be with any suitable method.

Accordingly, in one embodiment of the invention the pharmaceutical composition is formulated for intravenous administration. In some embodiments, the formulation is formulated for oral administration, rectal administration or subcutaneous administration.

The pharmaceutical composition may comprise additional pharmaceutically acceptable compounds, such as stabilizers, excipients and/or adjuvants.

Stabilizers for the present composition may comprise neutral salts, such as NaCL or KCl, sugars such as trehalose or amino acids. Preferably, the pharmaceutical composition is a buffered composition. Any pharmaceutically acceptable buffer system may be used, in particular phosphate buffer.

Suitable stabilizers, excipients and adjuvants are known to the person skilled in the art.

The pharmaceutical composition as defined above for managing/regulating blood coagulation can be administered to a subject in need thereof. The composition is particularly suitable for the treatment of mammals, preferably humans.

Accordingly, in one embodiment, the invention relates to a composition for managing and/or regulating blood coagulation of a subject as defined above, wherein the subject is a mamal. In preferred embodiments, said mammal is a human.

As it was found that ovomucoids are particularly suitable trypsin inhibitors for use in a method of managing and/or regulating In a further aspect, the invention relates to an ovomucoid for use in a method of managing or regulating blood coagulation.

In particular embodiments of the invention managing or regulating blood coagulation comprises regulating or modifying plasmin activity. In particular, the invention relates to an ovomucoid or fragment thereof for use in a method of managing or regulating blood coagulation, wherein the ovomucoid is a plasmin inhibitor.

In a particular embodiment, a method managing or regulating blood coagulation refers a method of managing or regulating blood coagulation is a method for treatment and/or prevention of hyperfibrinolysis associated bleeding.

The ovomucoid may be any ovomucoid, preferably, the ovomucoid or fragment thereof is a chicken egg, duck egg or turkey egg ovomucoid or a fragment thereof. In a particular embodiment, the invention relates to a duck egg ovomucoid or fragment thereof. In a preferred embodiment the ovomucoid is duck egg ovomucoid.

The ovomucoid may be a natural ovomucoid extracted from egg, or a recombinant ovomucoid.

The ovomucoid for use in a method for managing and/or regulating blood coagulation may be administered by any suitable administration. Preferably, the ovomucoid is administered intravenously. Alternatively, the ovomucoid may be administered orally, rectally or subcutaneously.

In the context of the present invention, all embodiments for the pharmaceutical composition comprising a trypsin inhibitor also apply to the ovomucoid for use in a method for regulating and/or managing blood coagulation.

In a further aspect, the invention relates to a method for managing and/or regulating blood coagulation, comprising administering a trypsin inhibitor to a subject in need thereof.

In a partwherein managing or regulating blood cagualtion comprises regulating or modifying plasmin activity. In particular, the method comprises inhibiting plasmin. In a particular embodiment, the method is a method for treatment and/or prevention of hyperfibrinolysis associated bleeding.

In one embodiment the invention relates to a method for managing and/or regulating blood coagulation the trypsin inhibitor is an ovomucoid or a fragment thereof. In particular embodiments, the method comprises administering an ovomucoid or fragment thereof selected from chicken egg ovomucoid, turkey egg ovomucoid and duck egg ovomucoid. In a particular preferred embodiment, the method comprises administering duck egg ovomucoid or a fragment thereof. In a particular embodiment, the method comprises administering duck egg ovomucoid.

In one embodiment, the invention relates to a method wherein the ovomucoid is administered subcutaneously, orally, rectally or intravenously. In a preferred embodiment, the method comprising an ovomucoid intravenously to a subject in need thereof.

The invention further relates to the following numbered items:

1. A pharmaceutical composition comprising a trypsin inhibitor for use in a method of managing or regulating blood coagulation in a subject.
2. The pharmaceutical composition for use according to item 1, wherein the trypsin inhibitor is a protein.
3. The pharmaceutical composition for use according to item 1 or 2, wherein managing or regulating blood coagulation comprises modifying or regulating plasmin activity.
4. The pharmaceutical composition for use according to any one of items 1 to 3, wherein the trypsin inhibitor is a plasmin inhibitor.
5. The pharmaceutical composition for use according to any one of items 1 to 4, wherein the trypsin inhibitor is an ovomucoid or a fragment thereof.
6. The pharmaceutical composition for use according to item 5, wherein the ovomucoid is duck egg ovomucid or a fragment thereof.
7. The pharmaceutical composition for use according to item 6, wherein the ovomucoid is a recombinant duck egg ovomucid or a fragment thereof.
8. The pharmaceutical composition for use according to any one of items 1 to 7, wherein managing or regulating blood coagulation refers to treatment and/or prevention of hyperfibrinolysis associated bleeding.
9. The pharmaceutical composition for use according to any one of items 1 to 8, wherein the composition comprises duck egg ovomucid for use in the treat-ment and/or prevention of hyperfibrinolysis associated bleeding.
10. The pharmaceutical composition for use according to any one of items 1 to 9, wherein the pharmaceutical composition is formulated for intravenous administration.
11. The pharmaceutical composition for use according to any one of items 1 to 10, wherein the pharmaceutical composition additionally comprises pharma-ceutically acceptable stabilizers and/or adjuvants.
12. The pharmaceutical composition for use according to any one of items 1 to 11, wherein the subject is a mamal.
13. The pharmaceutical composition for use according to item 12, wherein the mamal is a human.
14. An ovomucoid or a fragment thereof for use in use in a method of managing or regulating blood coagulation.
15. The ovomucoid or a fragment thereof for use according to item 14, wherein managing or regulating blood coagulation comprises regulating or modifying plasmin activity.
16. The ovomucoid or a fragment thereof for use according to item 14 or 15, wherein the ovomucoid is duck egg ovomucoid or a fragment thereof.
17. The ovomucoid or a fragment thereof for use according to any one of items 14 to 16, wherein the ovomucoid is a recombinant ovomucoid.
18. The ovomucoid or a fragment thereof for use according to any one of items 14 to 17, wherein a method of managing or regulating blood coagulation is a method for treatment and/or prevention of hyperfibrinolysis associated bleeding.
19. The ovomucoid for use according to any one of items 14 to 18, wherein the ovomucoid is administered intravenously.
20. A method for managing or regulating blood coagulation, comprising administering a trypsin inhibitor to a subject in need thereof.
21. The method for managing or regulating blood coagulation according to item 20, wherein the trypsin inhibitor is an ovomucoid or a fragment thereof.
22. The method for managing or regulating blood coagulation according to item 20 or 21, wherein managing or regulating blood cagualtion comprises regulat-ing or modifying plasmin activity.
23. The method for managing or regulating blood coagulation according to any one of items 20 to 22, wherein the trypsin inhibitor is duck egg ovomucoid or a fragment thereof.
24. The method for for managing or regulating blood coagulation according to any one of items 20 to 23, wherein the method is for treatment and/or prevention of hyperfibrinolysis associated bleeding.
25. The method for for managing or regulating blood coagulation according to any one of items 20 to 23, wherein the trypsin inhibitor is administered intravenously.

The following examples serve to illustrate the invention, however should not to be understood as restricting the scope of the invention.

### EXAMPLES

### Example 1 Bioequivalence Studies of Duck Egg Ovomucoid Compared to Anti-Thrombolytic Agents

This pilot bioequivalence study aims at investigating the effect of duck egg ovomucoid on the clotting system and assessed its activity against the known anti-thrombolytic agents (Trasylol® “aprotinin” and Cyklokapron” “transamic acid”) by means of the calibrated automated thrombogram (CAT). The CAT method is a functional sensitive assay which detects any hyper- or hypocoagulability in the clotting system, disorders in platelet function and follows up therapy with anti-haemophilia supplements, anticoagulants and antithrombotics.

Because duck egg ovomucoid is to be used under conditions of increased fibrinolysis, its behavior in the presence as well as in the absence of fibrin should be tested. It should be kept in mind here that the clotting mechanism in the presence and in the absence of fibrin are fundamentally different, in that in the absence of fibrin the mechanism is entirely chemical and in the presence of fibrin diffusion plays role as well.

### Results

### 1. Thrombin Generation (TG) Measured in the Absence of Fibrinogen

In a fibrinolytic patient fibrin is unstable. To mimic the extreme situation in which no fibrin is present, normal pooled plasma was treated with Ancrod, a clotting enzyme that does not affect the other coagulation factors, the clot was removed and TG then was measured in the defibrinated plasma in the absence and presence of duck egg ovomucoid (50-350 U/ml), Trasylol (50-350 U/ml) and Cyklokapron (1-8 mg/ml).

An optimal concentration of procoagulant phospholipids was used (4 μM). Two concentrations of tissue factor (TF) were added, 5 pM to study the classical extrinsic pathway and 0.5 pM to explore a pathway in which also the antihaemophilic factors (Factor VIII and IX) play a role.

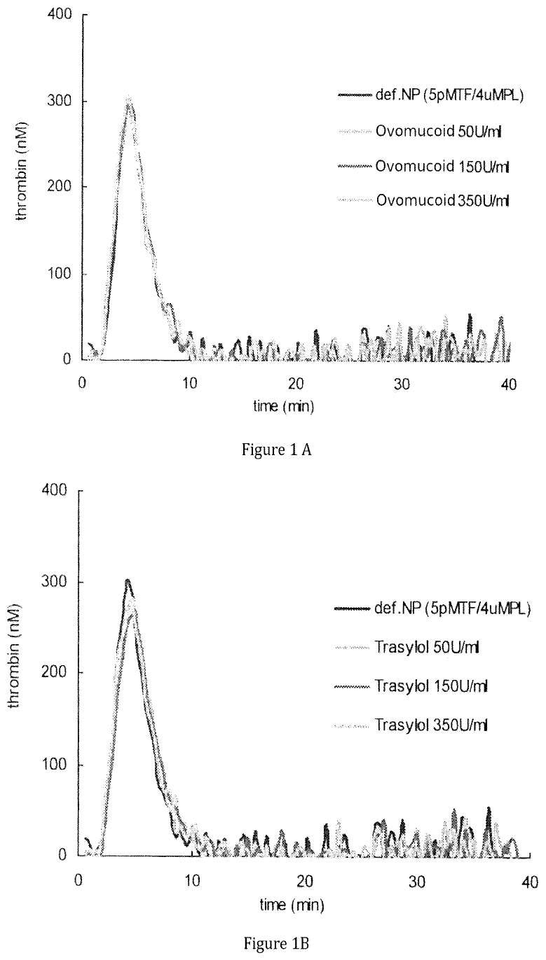

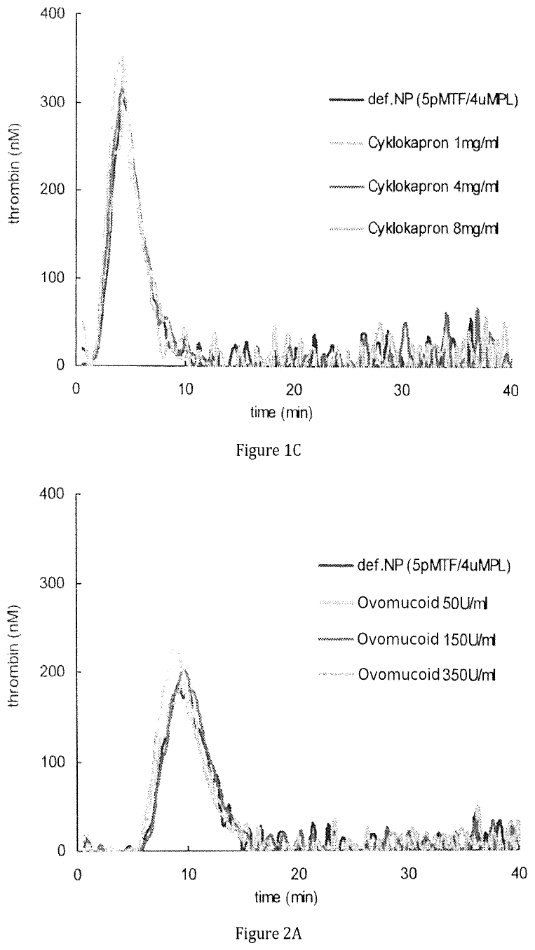

1) TG measured at 5 pM TF: when defibrinated normal plasma was treated with varying doses of the investigational products, extrinsic-thrombin generation showed no change at all doses tested and for all products added (FIG. 1a, b, c).

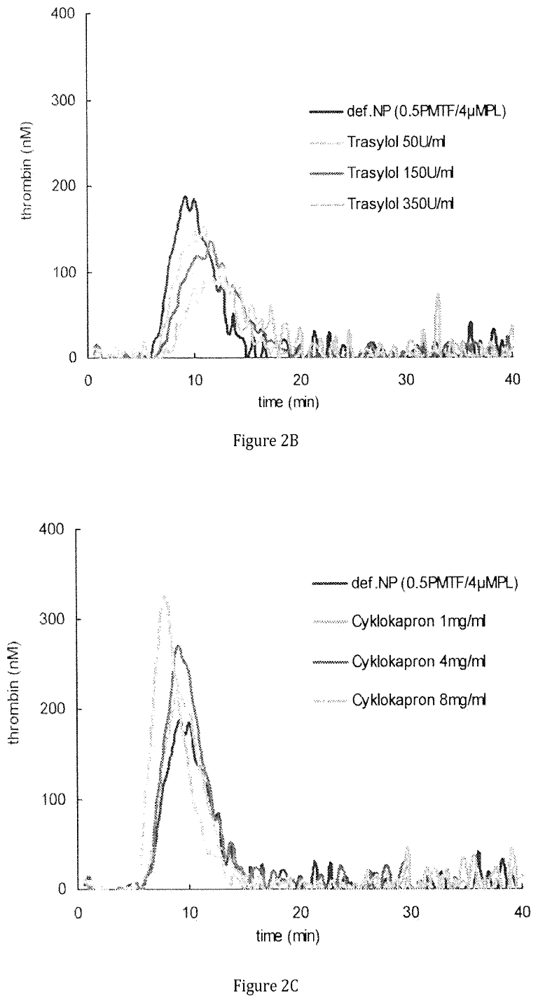

2) At lower TF concentration (0.5 pM), duck egg ovomucoid again showed no effect on TG while Trasylol at comparable doses inhibited thrombin generation dose-dependently, indicating a clear anticoagulant activity. Conversely, Cyklokapron at 0.5 pM TF increased the thrombin generation in a dose-dependent manner so that it can be considered as a procoagulant agent (FIG. 2a, b, c).

### 2. Thrombin Generation Measurement in the Presence of Endogenous Fibrinogen

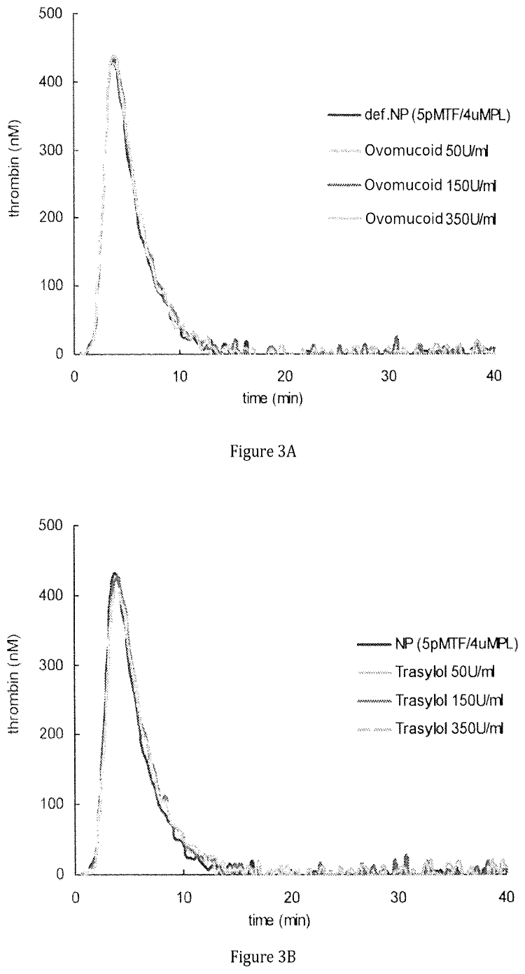

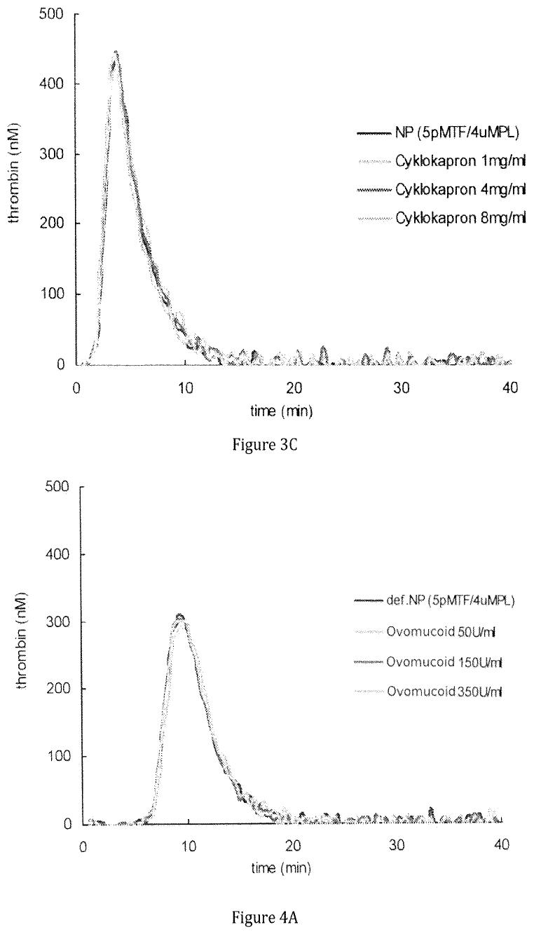

Normally fibrin (-ogen) is present during thrombin generation, therefore the above experiments were carried out in normal non-defibrinated plasma. Extrinsic-thrombin generation was conducted in normal plasma spiked with increasing amounts of duck egg ovomucoid (50-350 U/ml) and compared to Trasylol (50-350 U/ml) and to Cyklokapron (1-8 mg). It is seen as in defibrinated plasma that the three products showed hardly any activity at all doses tested, see FIG. 3a, b, c.

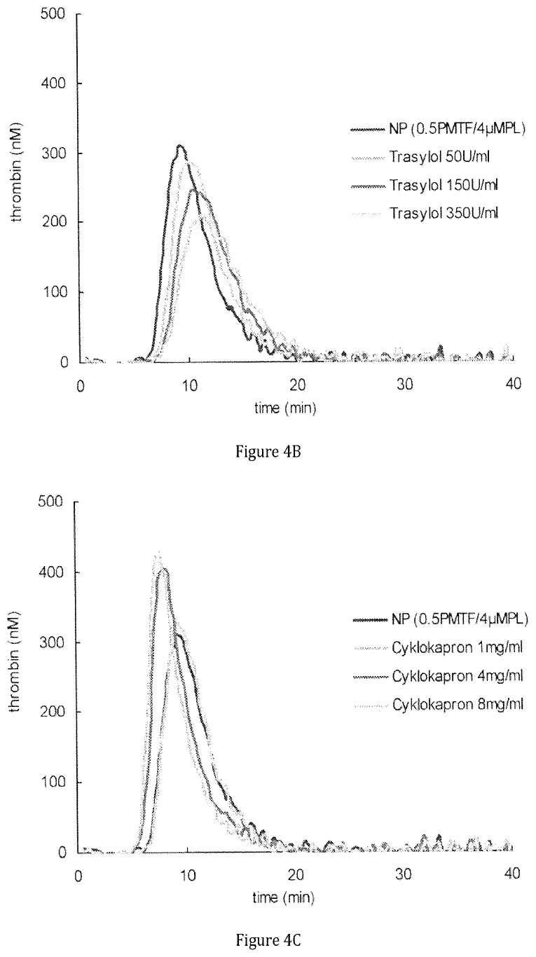

On the contrary, at low TF concentration, Trasylol and Cyklokapron exerted dose-dependently anticoagulant and procoagulant activities respectively. Duck egg ovomucoid nevertheless, as in the absence of fibrinogen, hardly affected the coagulation system at all of the doses used, FIG. 4a, b, c.

### CONCLUSION

This pilot study concludes that the new investgationla anti-thrombolytic (duck egg ovomucoid) showed hardly any activity on the clotting system in the absence and presence of fibrinogen compared to Trasylol and Cyklokapron. Trasylol exerted an anticoagulant activity which is probably located in the factor VIII and IX dependent section of the coagulation system, because it was seen only at low TF and independent of fibrinogen presence. Cyklokapron on the other hand showed a procoagulant activity which again, as with Trasylol, was observed at low TF (i.e. in the FVIII-IX section) and independent of the presence of fibrinogen.

Therefore the neutrality of Ovomin toward the coagulation system could be of clinical importance in its application as anti-fibrinolytic agent compared to the anticoagulant Trasylol and procoagulant cyklokapron. Further investigation on the anti-fibrinolytic effect of duck egg ovomucoid ex-vivo is warranted to confirm the present data.

### Example 2 Plasmin Inhibition Studies

In order to analyse plasmin inhibition a buffered plasmin solution was used. The solution was incubated with duck egg ovomucoid or cyklocapron as a control. The solutions were incubated at 37° C. and the chromogenic plasmin substrate S-2251 was added and plasmin activity was measured.

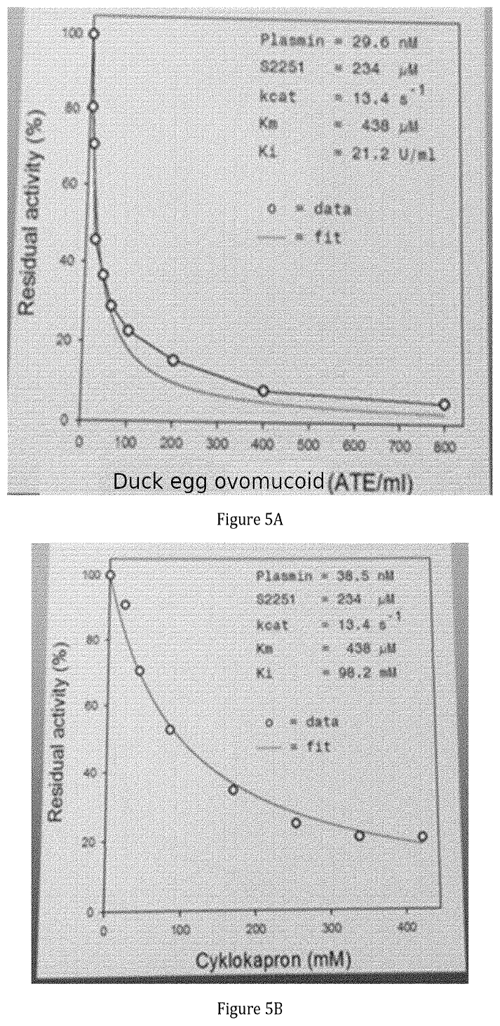

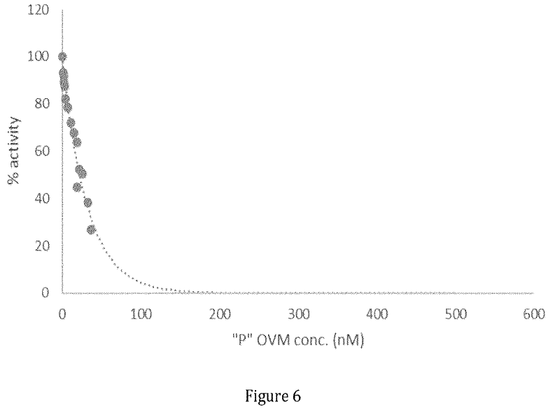

The results are shown in FIG. 5. It is clear that duck egg ovomucoid shows a comparable inhibitory effect on plasmin as cyklokapron.

### Example 3 Plasmin Inhibition Using Purified Duck Egg Ovomucoid

Pure ovomucoid was isolated from duck egg white using immobilized trypsin.

The obtained pure ovomucoid solution contained 0.163 mg of protein per ml. The purity of the sample was confirmed by HPLC analysis.

The kinetics of plasmin inhibition by ovomucoid were quantified by performing plasmin activity assay using different concentrations of pure ovomucoid. Loss of plasmin activity was estimated at each concentration of ovomucoid.

### Assay Setup:

| | Test | |
|---|---|---|
| Albumin buffer solution | 123 | μL |
| Diluted ovomucoid | 1 | μL |
| 2-fold diluted Plasmin | 1 | μL |
| Incubated for 10 min. at 37° C. | | |
| S-2251 | 25 | μL |

Read the absorbance at 405 nm for 20 min

Albumin buffer solution: 140 mM NaCl, 20 mM HEPES, 0.02% NaN3, 5 mg/mL BSA (pH 7.35)

S2251: 7 mg dissolved in 10 mL of H2O

Diluent: Take 1 mL of (0.2 M glycine+4 M urea pH 2.5)+add 40 μL of 2 M Tris HCl buffer pH 8. Use this diluent to generate the following dilutions of the purified ovomucoid.

| | Pure | | amount of | | | |
|---|---|---|---|---|---|---|
| | Ovomucoid | | protein | Concentration | | Residual |
| S. | solution | Diluent | added to | of ovomucoid | | activity |
| No. | (μL) | (μL) | assay (μg) | in assay (nM) | ΔA₄₀₅/min | (%) |
| 0 | 0 | 100 | 0 (Control) | 0 | 0.0157 | 100 |
| 1 | 2 | 98 | 0.00326 | 0.724 | 0.0146 | 92.99 |
| 2 | 4 | 96 | 0.00652 | 1.448 | 0.014 | 89.17 |
| 3 | 6 | 94 | 0.00978 | 2.173 | 0.0144 | 91.71 |
| 4 | 8 | 92 | 0.01304 | 2.897 | 0.0137 | 87.26 |
| 5 | 10 | 90 | 0.0163 | 3.622 | 0.0129 | 82.16 |
| 6 | 20 | 80 | 0.0326 | 7.244 | 0.0123 | 78.34 |
| 7 | 30 | 70 | 0.0489 | 10.866 | 0.0113 | 71.97 |
| 8 | 40 | 60 | 0.0652 | 14.488 | 0.0106 | 67.51 |
| 9 | 50 | 50 | 0.0815 | 18.111 | 0.01 | 63.69 |
| 10 | 60 | 40 | 0.0978 | 21.733 | 0.0082 | 52.22 |
| 11 | 70 | 30 | 0.1141 | 25.355 | 0.0079 | 50.31 |
| 12 | 80 | 20 | 0.1304 | 18.977 | 0.007 | 44.58 |
| 13 | 90 | 10 | 0.1467 | 32.6 | 0.006 | 38.21 |
| 14 | 100 | 0 | 0.163 | 36.222 | 0.0042 | 26.75 |

The results are illustrated in FIG. 6.

Plasmin activity was proportionately inhibited with increasing amount of ovomucoid in the assay mixture. Approximately 25 nM ovomucoid inhibited 50% of 80 nM plasmin activity present in the assay mixture.

1. A pharmaceutical composition comprising a trypsin inhibitor for use in a method of managing or regulating blood coagulation in a subject.

2. The pharmaceutical composition for use according to claim 1, wherein the trypsin inhibitor is a plasmin inhibitor.

3. The pharmaceutical composition for use according to any one of claim 1 or 2, wherein the trypsin inhibitor is an ovomucoid or a fragment thereof.

4. The pharmaceutical composition for use according to claim 3, wherein the ovomucoid is duck egg ovomucid or a fragment thereof.

5. The pharmaceutical composition for use according to any one of claims 1 to 4, wherein managing or regulating blood coagulation refers to treatment and/or prevention of hyperfibrinolysis associated bleeding.

6. The pharmaceutical composition for use according to any one of claims 1 to 5, wherein the composition comprises duck egg ovomucid for use in the treatment and/or prevention of hyperfibrinolysis associated bleeding.

7. The pharmaceutical composition for use according to any one of claims 1 to 6, wherein the pharmaceutical composition is formulated for intravenous administration.

8. The pharmaceutical composition for use according to any one of claims 1 to 7, wherein the subject is a mamal, preferably a human.

9. An ovomucoid or a fragment thereof for use in use in a method of managing or regulating blood coagulation.

10. The ovomucoid or a fragment thereof for use according to claim 9, wherein managing or regulating blood coagulation comprises regulating or modifying plasmin activity.

11. The ovomucoid or a fragment thereof for use according to claim 9 or 10, wherein the ovomucoid is duck egg ovomucoid or a fragment thereof.

12. The ovomucoid or a fragment thereof for use according to any one of claims 9 to 11, wherein a method of managing or regulating blood coagulation is a method for treatment and/or prevention of hyperfibrinolysis associated bleeding.

13. The ovomucoid for use according to any one of claims 9 to 12, wherein the ovomucoid is administered intravenously.
